(19) United States
(12) Patent Application Publication
(10) Pub. No.: US 2015/0290106 A1
(43) Pub. Date: Oct. 15, 2015

(54) Dentotropic Conjugates and Compositions and Methods of Use Thereof

(71) Applicant: BOARD OF REGENTS OF THE UNIVERSITY OF NEBRASKA, Lincoln, NE (US)

(72) Inventors: Dong Wang, Omaha, NE (US); Zhenshan Jia, Omaha, NE (US); Chen Fu, Omaha, NE (US)

(21) Appl. No.: 14/436,609

(22) PCT Filed: Oct. 17, 2013
(86) PCT No.: PCT/US13/65424

§ 371 Date: Apr. 17, 2015

Related U.S. Application Data
(60) Provisional application No. 61/715,524, filed on Oct. 18, 2012.

(51) Int. Cl. A61K 8/55; A61Q 11/02; A61Q 11/00

(52) U.S. Cl. CPC A61K 8/55; A61Q 11/00; A61Q 11/02; A61K 2800/592

(57) ABSTRACT

Compositions and methods for targeting agents, particularly a flavoring, fragrant, or cooling agent such as menthol, to tooth are provided.

This application claims priority under 35 U.S.C. §119(e) to U.S. Provisional Patent Application No. 61/715,524, filed on Oct. 18, 2012. The foregoing application is incorporated by reference herein.

### FIELD OF THE INVENTION

The present invention relates to dentotropic compounds. More specifically, the instant invention provides tooth targeting compounds which degrade and release a conjugated agent.

### BACKGROUND OF THE INVENTION

The success of delivering biologically active agents to the oral cavity has been largely limited by the fact that most of them do not have any tooth targeting specificity and do not maintain an effective concentration on the tooth surface.

Various delivery systems have been developed to maintain drug concentration in the oral cavity. These include bioadhesive tablets (Ali et al. (2002) Int. J. Pharm., 238:93-103; Giunchedi et al. (2002) Eur. J. Pharm. Biopharm., 53:233-9; Minghetti et al. (1997) Boll. Chim. Farm., 136:543-8), bioadhesive patches/films (Nafee et al. (2003) Acta Pharm., 53:199-212; Senel et al. (2000) Int. J. Pharm. 193:197-203), and bioadhesive gels and semisolids (Jones (1999) J. Pharm. Sci., 88:592-8; Schiff, T. (2007) J. Clin. Dent., 18:79-81; Vinholis et al. (2001) Braz. Dent. J., 12:209-13). Their mechanism of retention is based upon the bioadhesive polymers, which would adhere to the mucosal layer of the oral cavity. Though generally effective in maintaining drug presence in the oral cavity, these formulations provide the highest drug concentration at the mucosal epithelia instead of teeth surface. Local irritation at the site of adhesion and the uncomfortable sensation of a foreign object often lead to poor patient compliance (Mulhbacher et al. (2006) Int. J. Biol. Macromol., 40:9-14; Sudhakar et al. (2006) J. Control Release. 114:15-40). To bring direct and long lasting interaction of agents with teeth, varnish formulations have also been developed. They are generally applied by dental health practitioners during routine office visit. The long-term benefit of the periodic treatment, however, is limited due to the episodic nature dental caries.

Accordingly, improved means for the delivery and retention of agents in the oral cavity are desired. Indeed, menthol is currently used as a flavoring and freshening agent in numerous dental formulations, such as toothpaste and mouthwash. However, due to constant salivary flow within the mouth and to the lack of tooth and mouth anchoring properties of the substance, menthol cannot be retained for a significant amount of time in the oral cavity.

### SUMMARY OF THE INVENTION

In accordance with the instant invention, methods for treating, inhibiting, and or preventing an oral disease or disorder in a subject are provided. In a particular embodiment, the methods comprise administering to a subject a conjugate comprising an oral care agent conjugated to a tooth targeting moiety. In a particular embodiment, the tooth targeting moiety is pyrophosphate or a derivative thereof. In a particular embodiment, the oral care agent is menthol or a derivative thereof. The oral care agent may be linked directly to the tooth targeting moiety via a linker. The linker may be cleavable. In a particular embodiment, the linker is a lower alkyl.

In accordance with another aspect of the instant invention, conjugates and compositions for performing the methods of the instant invention are provided. In a particular embodiment, the conjugate comprises an oral care agent conjugated to a tooth targeting moiety. In a particular embodiment, the tooth targeting moiety is pyrophosphate or a derivative thereof. In a particular embodiment, the oral care agent is menthol or a derivative thereof. The oral care agent may be linked directly to the tooth targeting moiety via a linker. The linker may be cleavable. In a particular embodiment, the linker is a lower alkyl. Compositions encompassing at least one conjugate of the instant invention and at least one carrier are also provided. In a particular embodiment of the instant invention, the compositions may be selected from the group consisting of a mouthwash, toothpaste, dentifrice, film, dental floss coating, tooth powder, topical oral gel, mouth rinse, denture product, mouth spray, lozenge, oral tablet, chewable tablet, and chewing gum.

### BRIEF DESCRIPTIONS OF THE DRAWING

FIG. 1 provides a schematic for the synthesis of a menthol pyrophosphate derivative. Top: reaction of menthol with bromoacetic acid under catalysis. Bottom: reaction of menthol bromoacetate with a pyrophosphate derivative and subsequent ion exchange to produce final product.

FIG. 2 provides a graph of the menthol release after chloroform extraction over the course of 24 hours.

### DETAILED DESCRIPTION OF THE INVENTION

In accordance with the instant invention, conjugates comprising a tooth targeting moiety linked to a compound (e.g., a biologically active agent) are provided. The conjugated compounds include, without limitation, polypeptides, peptides, nucleic acids, synthetic and natural drugs, chemical compounds (e.g., small molecules), and lipids. The compound may be hydrophilic, hydrophobic, or amphiphilic. The conjugates of the instant invention exhibit several advantages over prior products. For example, the conjugates, by means of their degradation over time, allow for long-lasting delivery of the linked or conjugated compound on the tooth surface. Further, due to the degradation over time, the conjugate is temporary and will not permanently adversely affect or stain the teeth.

The compound conjugated to the tooth targeting moiety can be an “oral active agent” (an agent operable to treat, inhibit, and/or prevent a oral disease or disorder or provide a cosmetic benefit within the oral cavity). Examples of oral active agents include, without limitation: therapeutic agents (e.g., an antimicrobials, anti-inflammatory agents, etc.), analgesic agents (e.g., local analgesic agents), menthol and derivatives thereof, fragrant agents, flavoring agents, cooling agents, warming agents (e.g., cinnamon), fluoride, vitamins, nutraceuticals, tooth whitening agents, tooth coloring agents, bleaching or oxidizing agents (e.g., peroxide compounds), thickening agents, humectants (e.g., glycerin, propylene glycol, sorbitol, polypropylene glycol and/or polyethylene glycol), sweetening agents, and derivatives and analogs thereof. Examples of such agents can be found, for example, in U.S. Patent Application Publication No. 2006/0286044 and PCT/EP2005/009724, which are incorporated by reference herein. In a particular embodiment, the compound comprises a flavoring and/or fragrant agent. In a particular embodiment, the compound comprises methanol or a derivative thereof. Examples of derivatives of menthol include, without limitation: menthyl acetate, menthyl lactate, menthyl succinate, menthylacetic acid N-ethylamide, menthyl-4-hydroxypentanoate, menthyl-3-hydroxybutyrate, N,N-dimethyl menthyl succinamide, menthyl pyrrolidone carboxylate, monomenthyl glutarate, and menthyl salicylate.

The flavoring and/or fragrant agent may be a fruit (e.g., fruit oil or extract) or mint (e.g., mint oil or extract). Examples of flavoring and/or fragrant agents include, without limitation: limonene, spearmint, peppermint, wintergreen, sassafras, clove, borneol, sage, bay, parsley, eucalyptus, marjoram, cinnamon, citrus, lemon, lime, grapefruit, orange, menthol, carvone, anethole, vanillin, citric acid, licorice, cassia, cherry, strawberry, anise, apricot, banana, grape, apple, pineapple, bubblegum, fennel, lavender, neem, ginger, vanilla, pine, aloe, green tea, berry, jasmine, honey, cocoa, chocolate, coffee, cola, peanut, almond, cookie, watermelon, xylitol, sweeteners (e.g., dextrose, sucrose, maltose, dextrin, mannose, xylose, ribose, fructose, levulose, galactose, sorbitol, mannitol, xylitol, aspartame, or saccharin), and the like.

Coloring agents include, without limitation, pigments and dyes. Examples of orally acceptable coloring and whitening agents include FD&C dyes and pigments, fluorescent dyes (e.g., coumarin), talc, mica, magnesium carbonate, calcium carbonate, magnesium silicate, magnesium aluminum silicate, silica, titanium dioxide, zinc oxide, red, yellow, brown and black iron oxides, ferric ammonium ferrocyanide, manganese violet, ultramarine, titaniated mica, bismuth oxychloride, and mixtures thereof. The tooth coloring agent may be any single color or any combination of colors. The tooth coloring agent may be a non-natural color or fluorescent (e.g., for costume use (e.g., Halloween)). The tooth whitening agent or tooth coloring agent may increase the glowing, sparkling, luster, reflectivity or other visual effect of the teeth (e.g., pearling agents).

Therapeutic agents include, without limitation, antimicrobial compounds or peptides, anti-inflammatory agents (e.g., NSAIDs, glucocorticoids, kinase inhibitors, etc.), vaccines, probiotics, anti-plaque agents, chemoprophylactic agents, or remineralization agents.

As used herein, the term “cooling agents” refers to compounds which cool the body locally (e.g., oral cavity) or cause the body to perceive a lower temperature even if the temperature is not actually reduced. Examples of cooling agents include, without limitation, menthol and derivatives thereof, borneol, eucalyptus oil, eucalyptol, thymol, and methone glycerol acetal (MGA).

In a particular embodiment, the antimicrobial is effective against acid-tolerant and/or acid producing oral bacteria such as Lactobacilli and Streptococcus, particularly S. mutans. Antimicrobials include, without limitation, farnesol, chlorhexidine (chlorhexidine gluconate), apigenin, triclosan, and ceragenin CSA-13. In a particular embodiment, the antimicrobial is farnesol. In another embodiment, the antimicrobial is an antibiotic such as, without limitation, beta-lactams (e.g., penicillin, ampicillin, oxacillin, cloxacillin, methicillin, and cephalosporin), carbacephems, cephamycins, carbapenems, monobactams, aminoglycosides (e.g., gentamycin, tobramycin), glycopeptides (e.g., vancomycin), quinolones (e.g., ciprofloxacin), moenomycin, tetracyclines, macrolides (e.g., erythromycin), fluoroquinolones, oxazolidinones (e.g., linezolid), lipopeptides (e.g., daptomycin), aminocoumarin (e.g., novobiocin), co-trimoxazole (e.g., trimethoprim and sulfamethoxazole), lincosamides (e.g., clindamycin and lincomycin), metronidazole, polypeptides (e.g., colistin), and derivatives thereof.

The conjugates of the instant invention include at least one targeting moiety which is used to direct the compound specifically to teeth. Tooth targeting moieties are those compounds which preferentially accumulate in/on tooth rather than any other organ or tissue in vivo. Illustrative examples of tooth targeting moieties include, but are not limited to: pyrophosphate and derivatives thereof, triphosphate and derivatives thereof, bisphosphonates (e.g., alendronate), quaternary ammonium groups, peptides (e.g., peptides comprising about 2 to about 100 (particularly about 6) D-glutamic acid residues, L-glutamic acid residues, D-aspartic acid residues, L-aspartic acid residues, D-phosphoserine residues, L-phosphoserine residues, D-phosphothreonine residues, L-phosphothreonine residues. D-phosphotyrosine residues, and/or L-phosphotyrosine residues), tetracycline and analogs or derivatives thereof, sialic acid, malonic acid, N,N-dicarboxymethylamine, 4-aminosalicyclic acid, 5-aminosalicyclic acid, antibodies or fragments or derivatives thereof specific for tooth (e.g., Fab, humanized antibodies, and/or single chain variable fragment (scFv)), and analogs and derivatives thereof. In a particular embodiment, the targeting moiety comprises pyrophosphate or a derivative or salt thereof. The pyrophosphate (or derivative/salt thereof) may have the general formula:

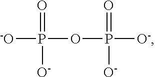

wherein one, two, or three O⁻ are bound by hydrogen or associated with a cation (e.g., Na⁺) and at least one O⁻ is attached to the linker or the tooth targeting moiety. Examples of derivatives or salts of pyrophosphate include, without limitation: sodium pyrophosphate, trisodium hydrogen pyrophosphate, sodium acid pyrophosphate, organic salts of pyrophosphate, tetrabutyl ammonium pyrophosphates, and tris(tetrabutylammonium) hydrogen pyrophosphate.

Alendronate, a bisphosphonate, has a high affinity for hydroxyapatite crystals (the main component of tooth enamel), and has been used clinically for the treatment of osteoporosis for many years (Russell, R. G. (2007) Pediatrics 119 (Suppl 2):S150-62). Pyrophosphate is known to adhere to hydroxyapatite, the main component of teeth and bone. It has been used in oral care products to provide abrasion and prevent tartar buildup and the in vivo instability of pyrophosphate allows for its eventual complete degradation into non-toxic byproducts after application. Therefore, by conjugating a compound such as menthol to a pyrophosphate, the compound (e.g., menthol) can be safely retained within the mouth (e.g., for sustained fresh breath after use of an oral care product).

The tooth targeting moiety may be linked to the compound via a bond or a linker. Generally, the linker is a chemical moiety comprising a covalent bond or a chain of atoms that covalently attaches the ligand to the surfactant. The linker can be linked to any synthetically feasible position of the tooth targeting moiety and the compound. The linker may be nondegradable or degradable (e.g., substantially cleaved). In a particular embodiment, the linker between the tooth targeting moiety and the compound is cleavable. For example, the linker may be cleaved upon a stimulus including, but not limited to, changes in pH, presence of a specific enzyme activity (i.e., the linker comprises an amino acid sequence cleavable by a protease), presence of reductases (i.e., linker comprises disulfide bond), changes in oxygen levels, or bacterial metabolites. In a particular embodiment, the linker may contain from 0 (i.e., a bond) to about 500 atoms, about 1 to about 100 atoms, about 1 to about 50 atoms, about 1 to about 25 atoms, or about 1 to about 10 atoms. The linker may also be a polypeptide (e.g., from about 1 to about 5). In a particular embodiment, the linker is a hydrocarbon, particularly an alkyl, particularly a lower alkyl. In a particular embodiment, the linker comprises an ester.

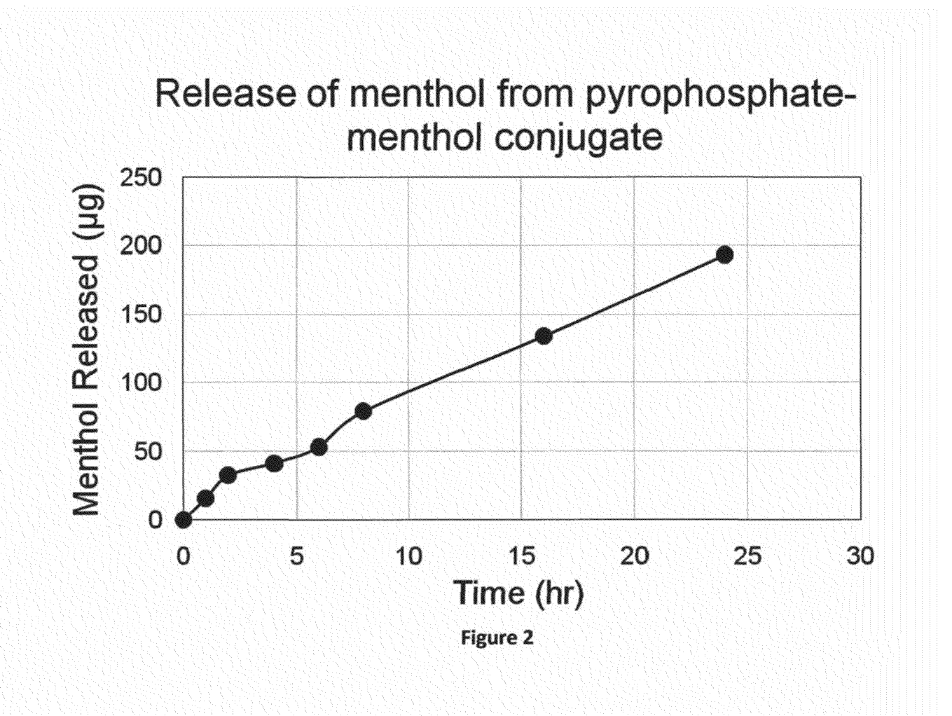

In a particular embodiment, the dentotropic compound comprises menthol conjugated to pyrophosphate. The menthol-pyrophosphate compound can bind to the surface of teeth and gradually degrade, thereby releasing menthol. The release of menthol over time provides the oral cavity with a sustained refreshing flavor and cool feeling. Accordingly, the menthol-pyrophosphate compound may be used to enhance the oral hygiene products by providing the consumer with a “clean” feeling after the use of a particular product, thereby significantly improving the new oral hygiene product. In a particular embodiment, the dentotropic compound is sodium 2-((1R,2S,5R)-2-isopropyl-5-methylcyclohexyloxy)-2-oxoethyl diphosphate (see, e.g., FIG. 2).

Compositions comprising at least one conjugate are also encompassed by the instant invention. The composition may further comprise at least one carrier (e.g., a pharmaceutically acceptable carrier). In a particular embodiment, the conjugate of the instant invention is used in an oral hygiene product. Examples of compositions as oral hygiene products include, without limitation: mouthwash, toothpaste, dentifrice, film, dental floss coating, tooth powder, topical oral gel, mouth rinse, denture product, mouth spray, lozenge, oral tablet, chewable tablet, and chewing gum. The compositions of the instant invention may comprise one or more additional oral care agents.

The compositions of the instant invention may be animal (e.g., pet) oriented. For example, the conjugates of the instant invention may be delivered via a chew to an animal. The chew may, for example, be coated and/or infused with the conjugates. As used herein, a “chew” is any toy, accessory, or foodstuff that is intended for chewing and/or gnawing by an animal. Chews may be made from animal products (e.g., hide, tendon or bone), synthetic products (e.g., plastics (e.g., nylon) or rubber), and plant products (e.g., rubber). In a particular embodiment, the chew is bone-shaped. The chew may be flavored with a flavoring that is appealing to the intended animal (e.g., chicken or beef flavoring).

The instant invention also encompasses methods of treating, inhibiting, and/or preventing oral diseases and disorders in a subject (e.g., humans or animals). The methods encompass the administration of at least one conjugate or composition of the instant invention to a subject in need thereof. Examples of oral diseases and disorders include, without limitation, caries, gingivitis, periodontitis, periodontitis-associated bone loss, dentin hypersensitivity, oral mucosal disease, oral mucositis, vesiculo-erosive oral mucosal disease, stained/discolored teeth, dry mouth, and halitosis.

The conjugates and compositions described herein will generally be administered to a patient as an oral hygiene product. The term “patient” as used herein refers to human or animal subjects. The conjugates may be employed therapeutically, under the guidance of a physician. In addition, the conjugates may also be used to deliver non-therapeutic compounds (e.g. cosmetic compounds or nutraceuticals).

The dose and dosage regimen of the compositions according to the invention that is suitable for administration to a particular patient may be determined by a physician considering the patient's age, sex, weight, general medical condition, and the specific condition for which the composition is being administered and the severity thereof. The physician may also take into account the route of administration of the composition, the pharmaceutical carrier with which the dentotropic compound is combined, and the dentotropic compound's biological activity.

Compositions of the instant invention may be administered orally. Pharmaceutical preparations for oral administration are known in the art. Pharmaceutical compositions containing a conjugate of the present invention as the active ingredient in intimate admixture with a pharmaceutical carrier can be prepared according to conventional pharmaceutical compounding techniques. The carrier may take a wide variety of forms depending on the form of preparation desired for administration, e.g., oral. In preparing the conjugate in oral dosage form, any of the usual pharmaceutical media may be employed, such as, for example, water, glycols, oils, alcohols, flavoring agents, preservatives, coloring agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Additionally, the conjugate of the instant invention may be administered in a slow-release matrix. For example, the conjugate may be administered in a gel comprising unconjugated poloxamers.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight or other conditions of the patient. Appropriate concentrations for alleviation of a particular pathological condition may be determined by dosage concentration curve calculations, as known in the art.

The pharmaceutical preparation may be administered at appropriate intervals, for example, at least once, twice a day or more until the pathological symptoms are reduced or alleviated, after which the dosage may be reduced to a maintenance level. The appropriate interval in a particular case would normally depend on the condition of the patient.

The compositions or conjugates of the instant invention may be used to treat and/or prevent caries. Treating caries may include administration of the compositions of the present invention to a subject suffering from caries for the purpose of reducing the amount of cariogenic bacteria such as Streptococcus mutans and/or for completely depleting Streptococcus mutans from the oral cavity, mouth, and/or teeth. The prevention of caries includes prophylaxis of caries. The compositions of the instant invention may be administered to subjects who have are at risk for encountering cariogenic bacteria such as Streptococcus mutans (e.g., have not encountered cariogenic bacteria and/or do not currently have cariogenic bacteria in the oral cavity). The compositions may be administered to infants or children for prophylaxis of caries since their oral cavity is normally free of Streptococcus mutans.

When used to treat and/or prevent oral disease or disorders, the conjugates of the instant invention may be contained within a composition comprising at least one orally acceptable carrier (i.e., a pharmaceutically acceptable carrier which can be used to apply the composition to the oral cavity in a safe and effective manner). As stated hereinabove, the composition may be in the form of a mouthwash, toothpaste, dentifrice (paste, liquid, or powder), dental floss coating, dental film, tooth powder, topical oral gel, mouth rinse, denture product, mouthspray, lozenge, oral tablet, chewable tablet, or chewing gum. Such compositions may further comprise other oral active agents such as, without limitation, chelating agents, fluoride, teeth whitening agents, tooth coloring agents (including non-natural colors), bleaching or oxidizing agents, cooling agent, vitamins, neutraceuticals, thickening agents, humectants, flavouring agents, fragrant agents, sweetening agents, and other antimicrobial agents.

### DEFINITIONS

The following definitions are provided to facilitate an understanding of the present invention:

The singular forms “a,” “an,” and “the” include plural referents unless the context clearly dictates otherwise.

“Substantial cleavage” occurs when at least 50% of the conjugates are cleaved, at least 75% of the conjugates are cleaved, at least 90% of the conjugates are cleaved, or at least 95% of the conjugates are cleaved.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), bulking substance (e.g., lactose, mannitol), excipient, auxiliary agent, filler, disintegrant, lubricating agent, binder, stabilizer, preservative or vehicle with which an active agent of the present invention is administered. Carriers can be sterile liquids, such as water, aqueous solutions, and oils, including those of petroleum, animal, vegetable or synthetic origin, such as peanut oil, soybean oil, mineral oil, sesame oil and the like. Water or aqueous solutions (e.g., aqueous saline solutions and aqueous dextrose and glycerol solutions) may be employed carriers for oral administration. In a particular embodiment, the carrier is an “orally acceptable carrier,” a material or combination of materials that are safe for use in the oral cavity (e.g., the cavity from the lips to the epiglottis). Preferably, the carrier does not substantially reduce the efficacy of the active materials of the present compositions. The composition of the present invention can be prepared, for example, in liquid form, or can be in dried powder form (e.g., lyophilized). Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy (Lippincott. Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, or nucleic acids, though they may be amino acids or dipeptides.

The term “alkyl,” as employed herein, includes straight, branched, and cyclic chain hydrocarbons containing 1 to about 20 carbons or 1 to about 10 carbons in the normal chain. The hydrocarbon chain of the alkyl groups may be interrupted with one or more oxygen, nitrogen, or sulfur. Each alkyl group may, optionally, be substituted, e.g., with 1 to 4 substituents. The term “lower alkyl” refers to an alkyl which contains 1 to 3 carbons in the hydrocarbon chain. The term “cyclic alkyl” or “cycloalkyl.” as employed herein, includes cyclic hydrocarbon groups containing 1 to 3 rings which may be fused or unfused. Cycloalkyl groups may contain a total of 3 to 20 carbons forming the ring(s), particularly 6 to 10 carbons forming the ring(s). Optionally, one of the rings may be an aromatic ring as described below for aryl. The cycloalkyl groups may also, optionally, contain substituted rings that includes at least one (e.g., from 1 to about 4) sulfur, oxygen, or nitrogen heteroatom ring members. Each cycloalkyl group may be, optionally, substituted, with 1 to about 4 substituents. Alkyl substituents include, without limitation, alkyl, alkenyl, halo (such as F, Cl, Br, I), haloalkyl (e.g., CCl₃ or CF₃), alkoxyl, alkylthio, hydroxy, methoxy, carboxyl, oxo, epoxy, alkyloxycarbonyl, alkylcarbonyloxy, amino, carbamoyl (e.g., NH₂C(═O)— or NHRC(═O)—, wherein R is an alkyl), urea (—NHCONH₂), alkylurea, aryl, ether, ester, thioester, nitrile, nitro, amide, carbonyl, carboxylate and thiol.

A “therapeutically effective amount” of a compound or a pharmaceutical composition refers to an amount effective to prevent, inhibit, or treat the symptoms of a particular disorder or disease.

As used herein, the term “subject” refers to an animal, particularly a mammal, particularly a human.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition (e.g., oral disease or disorder) resulting in a decrease in the probability that the subject will develop the condition.

As used herein, the term “nutraceutical” refers to a food or food part that provides medical or health benefits, including the prevention and treatment of disease. The nutraceutical may be a product isolated or purified from raw or unprocessed food materials.

As used herein, an “anti-inflammatory” refers to compounds which reduce the inflammatory response and/or are used for the treatment of an inflammatory disease or the symptoms associated therewith. Anti-inflammatory therapeutic agents include, without limitation, non-steroidal anti-inflammatory drugs (NSAIDs: e.g., aspirin, ibuprofen, naproxen, methyl salicylate, diflunisal, indomethacin, sulindac, diclofenac, ketoprofen, ketorolac, carprofen, fenoprofen, mefenamic acid, piroxicam, meloxicam, methotrexate, celecoxib, valdecoxib, parecoxib, etoricoxib, and nimesulide), corticosteroids (e.g., prednisone, betamethasone, budesonide, cortisone, dexamethasone, hydrocortisone, methylprednisolone, prednisolone, tramcinolone, and fluticasone), rapamycin, rho-kinase inhibitors, viral CC-chemokine inhibitor (vCCIs), glucocorticoids, steroids, beta-agonists, anticholinergic agents, methyl xanthines, sulphasalazine, dapsone, psoralens, proteins, peptides. DMARDs, glucocorticoids (e.g., dexamethasone), methotrexate, sulfasalazine, chloriquine, gold, gold salt, copper, copper salt, penicillamine, D-penicillamine, cyclosporine, lipoxins, resolvins, cox-2 inhibitors, MAP kinase inhibitors, caspase-1 inhibitors, JNK inhibitors, ERK inhibitors, Syk inhibitor, JAK inhibitors, and protectins. Anti-inflammatory therapeutic agents are also provided in The Pharmacological Basis of Therapeutics. Gilman et al., eds., McGraw-Hill Press and Remington's Pharmaceutical Science's, Easton: Mack Publishing Co.

As used herein, the term “analgesic” refers to an agent that lessens, alleviates, reduces, relieves, or extinguishes pain in an area of a subject's body (i.e., an analgesic has the ability to reduce or eliminate pain and/or the perception of pain without a loss of consciousness). Analgesics include opioid analgesics (e.g., codeine, dihydrocodeine, diacetylmorphine, hydrocodone, hydromorphone, levorphanol, oxymorphone, alfentanil, buprenorphine, butorphanol, fentanyl, sufentanyl, meperidine, methadone, nalbuphine, propoxyphene and pentazocine) and non-opiate analgesics (e.g., NSAIDs such as salicylates (e.g., aspirin, methyl salicylate, and diflunisal); arylalkanoic acids (e.g., indomethacin, sulindac, diclofenac, and tolmetin); N-arylanthranilic acids (e.g., fenamic acids, mefenamic acid, and mecflofenamate); oxicams (e.g., piroxicam and meloxicam); coxibs (e.g., celecoxib, rofecoxib, valdecoxib, parecoxib, and etoricoxib); sulphonanilides (e.g., nimesulide); naphthylalkanones (e.g., nabumetone); anthranilic acids (e.g., pyrazolidinediones and phenylbutazone); proprionic acids (e.g., fenoprofen, flurbiprofen, ibuprofen, ketoprofen, naproxen, and oxaprozin); pyranocarboxylic acids (e.g., etodolac); pyrrolizine carboxylic acids (e.g., ketorolac); and carboxylic acids. Analgesics are also provided in Goodman & Gilman's Pharmacologic Basis of Therapeutics.

The following example provides illustrative methods of practicing the instant invention, and is not intended to limit the scope of the invention in any way.

### Example

### Materials and Methods

### 1. Synthesis of (1R,2S,5R)-2-isopropyl-5-methylcyclohexyl 2-bromoacetate (Menthol Bromoacetate)

Menthol (1.56 g, 10 mmol) and a molar excess of bromoacetic acid (1.81 g, 13 mmol) were combined with 4-dimethylaminopyridine catalyst (DMAP; 0.24 g, 2 mmol). The DMAP also serves to suppress unwanted side reactions. Together, these substances were dissolved in 40 ml anhydrous dichloromethane (DCM). The solution was cooled down to 0° C. by an ice/water bath. N,N′-dicyclohexylcarbodiimide (DCC; 2.27 g, 11 mmol) was then added, creating a white precipitate of N,N′-dicyclohexylurea (DCU). The mixture was stirred for about two hours and then was filtered through filter paper. The filtrate was purified using column chromatography. More specifically, an LH-20 column was loaded with silica and packed with ethyl acetate (EA) and hexanes, 1:5 (volume-based). The eluted product was then evaporated of solvents. The final product yield was 2.5 g, or 91%. The results were confirmed via ¹H NMR spectra recorded on a Varian UNITY INOVA 500 NMR Spectrometer. ¹H-NMR (500 MHz, CDCl₃): δ 4.73 (td, J=11.22 Hz, 4.39 Hz, 1H), 3.81 (d, J=11.71 Hz, 1H), 3.79 (d, J=11.71 Hz, 1H), 2.01 (d, J=11.70 Hz, 1H), 1.91 (m, 1H), 1.70 (m, 1H), 1.68 (m, 1H), 1.50 (m, 1H), 1.43 (tt, J=12.21 Hz, 2.93 Hz, 1H), 1.06 (m, 1H), 1.03 (m, 2H), 0.92 (d, J=6.34 Hz, 3H), 0.90 (d, J=6.83 Hz, 3H), 0.77 (d, J=6.83 Hz, 3H) ¹³C-NMR (125 MHz, CDCl₃): δ 166.85, 76.41, 46.89, 40.42, 34.07, 31.34, 26.28, 26.09, 23.28, 21.95, 20.70, 16.18.

### 2. Synthesis of sodium 2-((1R,2S,5R)-2-isopropyl-S-methylcyclohexyloxy)-2-oxoethyl diphosphate (Menthol pyrophosphate derivative)

Menthol bromoacetate (415 mg, 1.5 mmol) was dissolved in 10 ml anhydrous acetonitrile (CH₃CN) and then cooled to 0° C. with ice-water bath. Tris(tetra-n-butylammonium) hydrogen pyrophosphate ((Bu₄N)₃HOP₂O₆; 2.7 g, 3 mmol) was dissolved in 5 ml anhydrous acetonitrile and then added dropwise to the methanol bromoacetate solution. The mixture was stirred at 0° C. for 1 hour under the protection of argon. Thin-layer chromatography confirmed the completion of this reaction. The solvent was evaporated and the residue was loaded on an LH-20 column with 40 g of Amberlite IR120, Na+ form, ion exchange resin. Distilled water was used to slowly elute the product. This process was repeated to ensure complete transformation of the tetra-n-butylammonium ion to sodium. The collected eluent was lyophilized. To rid the free sodium pyrophosphate, the sample was loaded onto a cellulose-packed column in a solvent of isopropyl alcohol:acetonitrile:water in a volume ratio of 4.5:2.5:3. The obtained eluent was lyophilized to give the pure final product: 567.7 mg, yield: 86.0%.

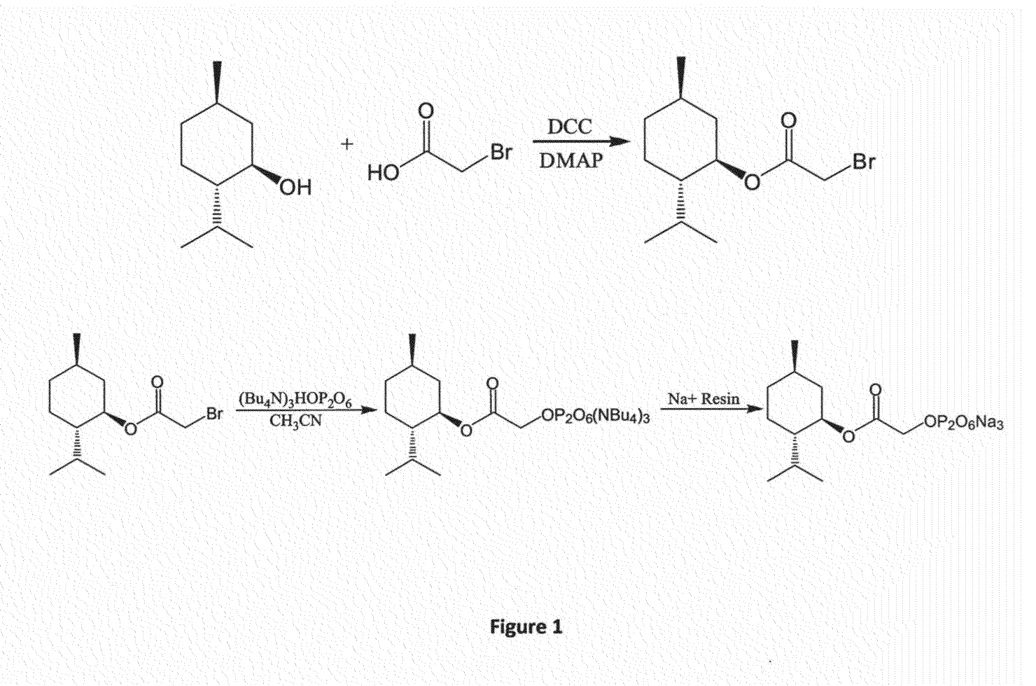

The synthesis scheme is depicted in FIG. 1.

¹H-NMR (500 MHz, D₂O): δ 4.71 (m, 1H), 4.55 (d, J=1.95 Hz, 1H), 4.53 (d, J=2.44 Hz, 1H), 1.99 (m, 1H), 1.84 (m, 1H), 1.69 (m, 2H), 1.45 (m, 2H), 1.08 (m, 3H), 0.89 (d, J=6.35 Hz, 3H), 0.87 (d, J=6.83 Hz, 3H), 0.74 (d, J=7.33 Hz, 3H) ¹³C-NMR (125 MHz, D₂O): δ 173.75, 79.59, 65.33, 48.92, 42.43, 36.04, 33.36, 28.16, 25.41, 23.74, 18.07 ³¹P-NMR (202 MHz, D₂O): δ −9.47 (d, J=20.75 Hz, 1P), −10.85 (d, J=20.75 Hz, 1P).

### 3. Model Tooth Surface Binding Experiment

Menthol-PPi (80 mg) was dissolved in deuterium oxide (D₂O, 16.1159 g). Two drops (34.7 mg) of acetone was added as an internal reference when using NMR spectroscopy. Four time intervals were chosen for this study, each with three samples for each interval. Into each of twelve microcentrifuge tubes was added 10 mg hydroxyapatite (HA). One by one, 1.0 g D₂O was added to the tubes and then agitated with a vortex mixer at high speed for the allotted amount of time. Each tube was then centrifuged at 12.000 rpm for one minute. The liquid was then transferred to NMR tubes for analysis. Three blank NMR samples without the influence of HA were prepared for reference, as well as three control samples without pyrophosphate (i.e., menthol only) with HA. The spectra of the blank samples were compared to those of the experimental samples by observing the relative integration areas of the acetone peak vs. the signal from one methyl group of menthol's isopropyl component. No binding is observed without pyrophosphate. The results of the experimental samples (Table 1) reveal that maximum binding takes place quickly, within one minute.

TABLE 1
Menthol-PPi/HA binding experiments.

| Time | HA-Bound |
|---|---|
| (min) | Conjugate (% ± σ %) |
| 1 | 27.56 ± 4.44 |
| 2 | 25.69 ± 0.77 |
| 5 | 28.30 ± 2.70 |
| 10 | 28.25 ± 3.04 |

An additional experiment was performed, changing the hydroxyapatite amount while keeping the vortex time fixed at 2 minutes for each sample, simulating the average time a person spends brushing his or her teeth. Menthol-PPi (80 mg) was dissolved in 16.14 g D₂O. Two drops (25.7 mg) of acetone were again added as internal reference. One gram of the menthol-PPi solution was added to the HA test tubes just prior to vortex agitation, as before. Samples were again analyzed via ¹H NMR. Table 2 shows the retention of the menthol-PPi to hydroxyapatite.

TABLE 2
Menthol-PPi binding experiments wherein the time was
fixed at two minutes, while the HA amount differed.

| HYDROXYAPATITE | HA-BOUND |
|---|---|
| (mg) | CONJUGATE (% ± σ %) |
| 20 | 33.88 ± 6.32 |
| 30 | 45.04 ± 1.34 |
| 40 | 54.51 ± 3.65 |

### 4. Release of Menthol in Human Saliva

Three equal portions of sodium 2-((1R,2S,5R)-2-isopropyl-5-methylcyclohexyloxy)-2-oxoethyl diphosphate (10.0 mg. 0.0227 mmol) was dissolved in fresh saliva (2 ml), respectively. The three bottles were shaken in a water-bath incubator at 37° C. and taken out of the incubator after 2 hours, 6 hours, and 20 hours. The samples were extracted with chloroform (2 ml) respectively. The chloroform solution was centrifugalized and 0.4 ml chloroform solution was taken out and mixed with 0.4 mL neomenthol solution (4 ppm in acetonitrile). Menthol in the three samples was analyzed with GC·MS. Calibration: Neomenthol (10.0 mg) and menthol (10 mg) were dissolved in 100 ml acetonitrile. 10 ml of the solution was diluted into 250 ml acetonitrile to give a solution with the menthol and neomenthol being 4 ppm respectively.

TABLE 3
The integration ratio is 0.9995. Accordingly, the amount
of menthol can be calculated by comparing the relative
integration area of neomenthol and menthol.

GC-MS integration

| | neomenthol | menthol |
|---|---|---|
| a | 50031 | 49566 |
| b | 50873 | 51092 |
| c | 49855 | 50170 |
| Integration area sum | 150759 | 150828 |

| | |
|---|---|
| Ratio | 0.9995 |
| (neomenthol/menthol) | |

TABLE 4
GC-MS assay of released menthol.

| | Rel. | Integration Area | | | [M] | Amt. | Rel. |
|---|---|---|---|---|---|---|---|
| | Time | N | M | Ratio | (ppm) | (μg) | (μg) |
| Blank | 0 | 24169 | Trace | 0 | 0 | 0 | 0 |
| Sample 1 | 2.5 | 16106 | 74555 | 4.629 | 9.258 | 7.4 | 37.0 |
| Sample 2 | 6 | 21105 | 243004 | 11.514 | 23.028 | 18.4 | 92.0 |
| Sample 3 | 20 | 26275 | 885813 | 33.713 | 67.426 | 53.9 | 269.5 |

Rel. time: Release time (hours); N: neomenthol; M: menthol; [M]: menthol concentration (integration area ratio × 2 ppm); Amt.: absolute amount (menthol conventration × 0.8 ml); Rel.: release amount (absolute amount (μg) × 2 ml/0.4 ml). The total menthol amount in sodium 2- ((1R,2S,5R)-2-isopropyl-5-methylcyclohexyloxy)-2-oxoethyl diphosphate (10.0 mg, 0.227 mmol) is 3.54 mg. Release percentages: Sample 1 = 1.05%; Sample 2 = 2.60%; Sample 3 = 7.61%.

Into several 15-mL glass vials were placed 10 mg of menthol-PPi and 2 mL of fresh saliva. The saliva had been centrifuged to remove any free particles, then filtered through a 0.45 μm syringe filter to rid bacteria. It was then diluted 1:1 by volume with phosphate-buffered saline (PBS). Each sample was placed in a water bath incubator at 37° C., shaking at 40 rpm, for a specific amount of time over the course of 24 hours. One sample was withdrawn at each time interval, and vortexed for 10 seconds after direct addition of 2 mL of chloroform. The mixture was transferred to microcentrifuge tubes, after which each tube was centrifuged at 12,000 rpm for one minute to separate the chloroform phase from the water (saliva/PBS) phase. The organic layer was removed, and 0.5 mL was transferred to 2-mL glass vials with septum caps for gas chromatography/mass spectrometry (GC/MS) analysis using an Agilent 6890 Series gas chromatography system with a 7683 Series injector, coupled with a 5973 Series mass selective detector. To the 0.5 mL of chloroform and menthol solution was added 0.5 mL of a 20 ppm (μg/mL) of neomenthol solution in acetonitrile. The neomenthol appears as a fairly well-separated peak with a retention time just before that of menthol, making for an ideal internal reference on GC/MS spectra. Additionally, it has been confirmed that the menthol and neomenthol peaks do not interfere with other substances that may be present in the chloroform extraction. After GC/MS analysis of each sample, the total amount of menthol released was calculated using the integration areas of menthol and neomenthol, simplified into a ratio. Each ratio was then multiplied by 10 ppm to account for the 2-fold dilution of the 0.5 mL of the 20 ppm internal standard. This gave the menthol concentration in ppm. The absolute amount, in μg, of menthol in each GC/MS vial was determined by multiplying the concentration by 1 mL, the total amount of solution in the vial. Then, the release amount was calculated by taking the previous value and multiplying it by a factor of 4, since the original sample was 2 mL. FIG. 2 provides the results.

It is evident that the menthol-PPi conjugate binds to HA powder very quickly. Further experiments include the use of Raman spectroscopy to confirm the binding of the substance onto HA discs, as well as on genuine human tooth sections. The release profile of the menthol from the pyrophosphate is linear in nature. Menthol conjugated to a pyrophosphate derivative clearly has a wide variety of application in dentifrice formulations. Its adherence to the tooth surface and its biodegradable nature make it an ideal agent for use in dental hygiene products.

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A compound comprising an oral active agent conjugated to pyrophosphate or a derivative thereof,
wherein said oral care agent is linked directly to said pyrophosphate or via a linker, wherein said linker is a lower alkyl.

2. The compound of claim 1, wherein said oral care agent comprises menthol or a derivative thereof.

3. The compound of claim 1, wherein said pyrophosphate derivative is a pyrophosphate salt.

4. The compound of claim 1, wherein said compound is sodium 2-((1R,2S,5R)-2-isopropyl-5-methylcyclohexyloxy)-2-oxoethyl diphosphate.

5. A composition comprising at least one compound of claim 1 and at least one carrier.

6. The compositions of claim 5, further comprising at least one additional oral care agent.

7. The composition of claim 5, wherein said composition is selected from the group consisting of mouthwash, toothpaste, dentifrice, film, dental floss coating, tooth powder, topical oral gel, mouth rinse, denture product, mouth spray, lozenge, oral tablet, chewable tablet, chew, and chewing gum.

8. A method for treating, inhibiting, or preventing an oral disease or disorder in a subject, said method comprising administering to said subject a composition of claim 5.

9. The method of claim 8, wherein said oral disease or disorder is halitosis.
